(19) United States
(12) Patent Application Publication
(10) Pub. No.: US 2016/0042637 A1
(43) Pub. Date: Feb. 11, 2016

(54) Drone Safety Alert Monitoring System and Method

(71) Applicant: Clandestine Development LLC, Arlington Heights, IL (US)

(72) Inventor: Peter Cahill, Arlington Heights, IL (US)

(73) Assignee: Clandestine Development, LLC

(21) Appl. No.: 14/824,011

(22) Filed: Aug. 11, 2015

Related U.S. Application Data
(60) Provisional application No. 62/035,762, filed on Aug. 11, 2014.

(51) Int. Cl. G08B 25/10; H04W 76/02; G08G 5/00; H04W 4/02

(52) U.S. Cl. CPC G08B 25/10; H04W 4/02; H04W 76/023; G08G 5/0078; G08G 5/0069

(57) ABSTRACT

A wearable safety alarm system includes one or more processors to receive identifying information and transmit the identifying information to an alarm response server from a mobile computing device, receive, by the mobile computing device, a unique identifier that identifies the identifying information in database associated with the alarm response server, receive a trigger of an alarm notification by one of a wearable device and the mobile computing device, determine a current location of the mobile computing device, transmit an alarm notification message to the alarm response server, the alarm notification message including the current location of the mobile computing device and the unique identifier, and transmit the current location of the mobile computing device to at least one drone.

### RELATED APPLICATIONS

This application claims the benefit of priority to U.S. Patent Application No. 62/035,762, filed Aug. 11, 2014, which is hereby incorporated herein by reference.

### FIELD

The present systems and methods relate generally to systems and methods for monitoring by a drone or unmanned aerial vehicle (UAV) that is triggered by an alarm notification message sent by a wearable device and/or a mobile computing device. More particularly, the systems and methods receive an alarm trigger and send an alarm notification message, including location information and a unique identifier representing identifying information, to a server. The server sends the location information to the drone. The drone travels to a location using the location information and begins monitoring the location.

### BACKGROUND

Mobile computing devices have gradually become a ubiquitous part of daily life. Traditionally, a mobile computing device such as a smartphone may be carried on a person in a pocket, a purse, a briefcase, a backpack, a messenger bag, etc. In other situations, the mobile computing device may be located nearby a person, such as on a table or in a car. In nearly all of these instances, users of smartphones and tablets have access to a portable device that is capable of communicating with others, capable of executing applications, and capable of sending and receiving information to other devices.

However, when a life threatening emergency strikes, it may not be possible to dial “911” and/or reach out for help as quick as necessary because the mobile computing device may not be within arm's reach and/or may be inaccessible. In other dangerous situations, even if a person is able to dial “911” and/or reach out for help, the person may not be able to relay information during a telephone call for a variety of reasons, e.g., an incapacitating injury or an attacker/intruder is nearby.

While mobile computing devices provide users the ability to communicate with others and reach out for help in the event of an emergency, it may be difficult or impossible to efficiently and accurately provide critical information to an emergency dispatch center when time is of the essence.

In addition, after the emergency dispatch center is notified of the emergency, first responders may have to travel to the person to provide assistance. While the first responders attempt to arrive as soon as possible, there is typically a period of time before the first responders are able to arrive. During this period of time, valuable evidence may be lost.

Accordingly, to meet these needs and others, there is a need for systems and methods as described herein.

### SUMMARY

Briefly described, aspects of the present disclosure generally relate to methods and systems for monitoring by a drone or unmanned aerial vehicle (UAV) that is triggered by an alarm notification provided through an application provided through a mobile device. As used throughout the present disclosure, a mobile device may be any mobile computing platform, including a smartphone, a tablet computer, a wearable device, etc.

In one aspect, a user provides identifying information to a wearable safety application executed by a wearable device and/or a mobile safety application executed by a mobile computing device. The wearable device and/or the mobile computing device send the identifying information to an alarm response server and the alarm response server stores the identifying information in a database. The alarm response server associates the identifying information with a unique identifier and sends the unique identifier to the wearable device and/or the mobile computing device. If an emergency occurs, the user may trigger the wearable safety application and/or the mobile safety application. The wearable safety application and/or the mobile safety application send an alarm notification message including location information and the unique identifier to the alarm response server. The alarm response server determines one or more personal safety answering points (PSAP) based on the location information. If the alarm notification is verified, e.g., the alarm notification is not a false alarm, the alarm response server sends the location information and the identifying information to a call center server associated with the one or more PSAPs for further action by emergency responders.

In addition, the alarm response server sends the identifying information and the location information to one or more drones. The one or more drones travel to a location based on the location information and begins monitoring. In addition, the call center server also may send the location information and the identifying information to one or more lifelines, e.g., a person to contact in the event of an emergency.

In one aspect, a drone safety alert monitoring system includes one or more processors to receive identifying information and transmit the identifying information to an alarm response server from a mobile computing device, receive, by the mobile computing device, a unique identifier that identifies the identifying information in database associated with the alarm response server, receive a trigger of an alarm notification by one of a wearable device and the mobile computing device, determine a current location of the mobile computing device, transmit an alarm notification message to the alarm response server, the alarm notification message including the current location of the mobile computing device and the unique identifier, and transmit the current location of the mobile computing device to at least one drone.

There are numerous examples in which the features and functions of the present subject matter may be embodied. And the solutions provided herein may be applied in various use contexts. It is understood that aspects of the present disclosure may provide police and other emergency responders with a system that supplements or compliments their work. In one example, upon the issuance of an alarm notification by a police officer (e.g., though a mobile or wearable device, including a computer in a police car), location information is sent to an alarm response server, which communicates the location information to a drone, which is deployed to the location to capture monitoring information (i.e., photographs, video, audio, etc.) at the location. When at the location, the drone can start to track and follow an object from the location. For example, a police officer may issue an alarm notification for a location at which a car has been pulled over. At the scene, the officer may place a target on the car that has been pulled over. The drone may identify the target as the object to follow. Then, if the car starts moving, the drone may follow the car, and target, without further instruction from the alarm response server or other outside party. Using a target the drone can follow moving objects without the need for further instruction.

These and other aspects, features, and benefits of the present disclosure will become apparent from the following detailed written description in conjunction with the accompanying drawings, although variations and modifications thereto may be implemented without departing from the spirit and scope of the novel concepts of the disclosure.

### BRIEF DESCRIPTION OF THE DRAWINGS

The accompanying drawings illustrate embodiments of the disclosure and, together with the written description, serve to explain the teachings, principles, and solutions provided by the disclosure. Wherever possible, the same reference numbers are used throughout the drawings to refer to the same or similar elements across the various embodiments.

FIG. 1 illustrates a block diagram of a drone safety alert monitoring system according to an example embodiment.

FIG. 2 illustrates example information in an alarm response (PSAP) database according to an example embodiment.

FIG. 3 is a flowchart illustrating a process for monitoring by the drone safety alert monitoring system according to an example embodiment.

FIG. 4 illustrates a block diagram of an example computer device for use with the example embodiments.

FIGS. 5-7 illustrate example screenshots of a mobile safety application executed by a mobile computing device according to an example embodiment.

FIG. 8 illustrates a perspective view of a drone according to an example embodiment.

FIG. 9 illustrates a perspective view of a wearable device according to an example embodiment.

FIG. 10 illustrates another a perspective view of a wearable device according to an example embodiment.

FIG. 11 illustrates a command center graphical user interface (GUI) according to an example embodiment.

### DETAILED DESCRIPTION

For the purpose of promoting an understanding of the principles of the present disclosure, reference will now be made to the embodiments illustrated in the drawings, and specific language will be used to describe the same. It will, nevertheless, be understood that no limitation of the scope of the disclosure is intended; alterations and further modifications of the described and illustrated embodiments, and further applications of the principles of the disclosure as illustrated therein, are contemplated as would normally occur to one skilled in the art to which the disclosure relates.

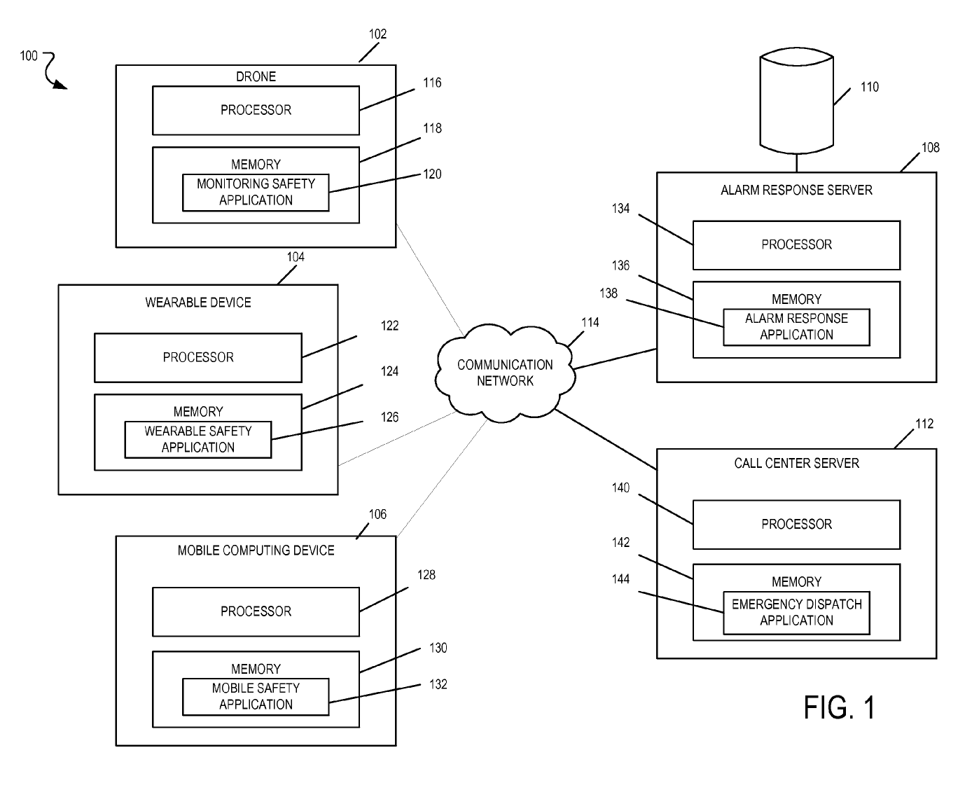

FIG. 1 illustrates a block diagram of a drone safety alert monitoring system 100 according to an example embodiment. According to an aspect of the present disclosure, the drone safety alert monitoring system 100 includes one or more drones 102. The drone safety alert monitoring system 100 further includes one or more optional wearable devices 104, one or more mobile computing devices 106, one or more alarm response servers 108, one or more databases 110, one or more call center servers 112, and a communication network 114. The one or more computing devices communicate and coordinate their actions by passing messages over the communication network 114. The communication network 114 can be one or more of the Internet, an intranet, a cellular communications network, a WiFi network, a packet network, or another wired or wireless communication network. As an example, the one or more computing devices communicate data in packets, messages, or other communications using a common protocol, e.g., Hypertext Transfer Protocol (HTTP) and/or Hypertext Transfer Protocol Secure (HTTPS). As an example, the drone safety alert monitoring system 100 may be a cloud-based computer system or a distributed computer system.

The one or more computing devices may communicate based on representational state transfer (REST) and/or Simple Object Access Protocol (SOAP). As an example, a first computer (e.g., a client computer) may send a request message that is a REST and/or a SOAP request formatted using Javascript Object Notation (JSON) and/or Extensible Markup Language (XML). In response to the request message, a second computer (e.g., a server computer) may transmit a REST and/or SOAP response formatted using JSON and/or XML.

The embodiments described herein may be based on Oauth, an open standard for authorization. Oauth allows producers of web services to grant third-party access to web resources without sharing usernames and/or passwords. In this case, the web resources may be the one or more drones 102, the one or more alarm response servers 108, the one or more databases 110, and the one or more call center servers 112. Oauth provides one application with one access token providing access to a subset of web resources on behalf of one user, similar to a valet key. In particular, the embodiments may be related to Oauth 2.0. While discussed in the context of Oauth, the present disclosure is not limited to Oauth.

The drone safety alert monitoring system 100 may be deployed or located at a particular site including a city, a town, a college campus, a corporate campus, outdoor venue, e.g., a concert venue, an indoor venue, e.g., an arena, and other locations. The drone safety alert monitoring system 100 may include one or more hangars to house the one or more drones 102. In one example, a college campus may have a single hangar housing the one or more drones 102. In another example, the one or more hangars may be distributed throughout the college campus. Each hangar may be located equidistant from other hangars, e.g., each hangar may each cover a particular grid on the particular site. However, each hangar also may be located in a particular location at the particular site based on previously reported emergencies and/or population density. As an example, the particular site may include four grids each having an equal size of 1000 feet×1000 feet. One hangar may be located in the center of each grid. Each hangar may house one or more drones 102 to quickly and efficiently service any particular location in each grid.

Each hangar may be outfitted with one or more alternating current (AC) power sockets and one or more chargers for charging the one or more batteries of the drone 102. As an example, the drone 102 may be housed in a hangar located on a roof of a building, a garage, or another location.

FIG. 1 illustrates a block diagram of the drone 102 according to an example embodiment. The drone 102 may be a computer having one or more processors 116 and memory 118, including but not limited to an unmanned aerial system (UAS) and an unmanned aerial vehicle (UAV). The drone 102 is not limited to an unmanned aircraft device or a UAV and may be other types of unmanned vehicles. The drone 102 may be an unmanned ground vehicle (UGV) having wheels, legs, or a continuous track, an unmanned vehicle traveling on rails, e.g., an unmanned train, an unmanned boat, and an unmanned hovercraft, among other vehicles. As an example, the drone 102 may be an autonomous or remote-controlled vehicle, an autonomous or remote-controlled car, an autonomous or remote-controlled train, an autonomous or remote-controlled boat, or an autonomous remote-controlled hovercraft, among other vehicles. However, for purposes of clarity, the majority of the description provided herein refers to the drone 102 as being an UAV.

The flight and operation of the drone 102 may be controlled autonomously by the one or more processors 116, another computer (e.g., the one or more alarm response servers 108 or another mobile computing device), and/or by one or more users via a remote control. The drone 102 may further include one or more cameras 103, one or more light sources, one or more microphones 105, one or more sensors including a gyroscope, an accelerometer, a magnetometer, an ultrasound sensor, an altimeter, an air pressure sensor, a motion sensor, and other sensors, one or more rotors, one or more motors, and one or more batteries for powering the drone 102. The camera 103 may be a high-definition camera capable of recording high-definition video (e.g., any video image with more than 480 horizontal lines and/or captured at rates greater than 60 frames per second). The camera 103 may include analog zoom and/or digital zoom and may zoom in or out during operation. The camera 103 also may be a thermal vision camera or a night vision camera. The drone 102 may be battery-powered and/or powered by another source, e.g., gasoline. The drone 102 may have a hull that comprises carbon fiber components, plastic components, metal components, and other components.

The drone 102 may communicate with another drone 102, the wearable device 104, the mobile computing device 106, the alarm response server 108, and/or the call center server 112 using at least one of Bluetooth, WiFi, a wired network, a wireless network, and a cellular network. According to an example embodiment, at least the drone 102 and the mobile computing device 106 may communicate wirelessly.

The drone 102 reverse geocodes a current location of the drone 102 using global positioning system (GPS) hardware. The GPS hardware communicates with a GPS satellite-based positioning system. The GPS hardware may be an assisted GPS system, e.g., A-GPS or aGPS, or may be a standalone GPS. Standalone GPS only uses radio signals from the satellite-based positioning system. An assisted GPS system uses network resources available to the drone 102 to locate and use the satellite-based positioning system in poor signal conditions, such as in a city where signals bounce off of buildings or pass through walls or tree cover.

The one or more processors 116 may process machine/computer-readable executable instructions and data, and the memory 118 may store machine/computer-readable executable instructions and data including one or more applications, including a monitoring safety application 120. The processor 116 and memory 118 are hardware. The memory 118 includes random access memory (RAM) and non-transitory memory, e.g., a non-transitory computer-readable medium such as one or more flash disks or hard drives. The non-transitory memory may include any tangible computer-readable medium including, for example, magnetic and/or optical disks, flash drives, and the like.

The monitoring safety application 120 may be a component of an application and/or service executable by the drone 102. For example, the monitoring safety application 120 may be a single unit of deployable executable code. The monitoring safety application 120 may also be one application and/or a suite of applications for monitoring a person that triggers an alarm notification. In a primary example, the monitoring safety application 120 receives a location of the person, determines a route for the drone 102 to fly to the person using the GPS hardware, routes the drone 102 to the person based on the route, and monitors the person using video, photographs, and/or audio from the camera 103.

For example, upon receipt of the alert notification, the drone 102 receives the location and determines a shortest and/or quickest route to the location. The route also may be determined by another computing device and transmitted to the drone 102. As an example, the alarm response server 108 may determine the route. The route may be determined based on weather conditions and obstacles including buildings, trees, power lines, and other obstacles.

After the route is determined, the drone 102 takes off and may travel at a first particular altitude and a particular speed to the location. The drone 102 may also travel at a variable altitude that could change during the flight and a variable speed that could change during the flight. The drone 102 may pass through one or more waypoints on the route to the destination. The waypoints may be automatically assigned or may be assigned by an operator or user before or during flight. The waypoints may be used to avoid obstacles, avoid a populated area, or for another reason. As an example, the particular altitude may be 50 feet, 100 feet, 200 feet, 1000 feet, 2000 feet, and other altitudes.

The drone 102 travels to the location based on the route and upon arrival the drone 102 begins monitoring. Once the drone 102 arrives at the location, the drone 102 may hover at a second particular altitude above the person for a particular period of time. The second particular altitude may be the same altitude as the first particular altitude or a different altitude from the first particular altitude. The video, photographs, and/or the audio may be based on an aerial view of the person. In another embodiment, the drone 102 may land at the location or near the location and the video, photographs, and/or the audio may be based on a terrestrial view of the person.

The monitoring safety application 120 determines and builds one or more data structures comprising a three-dimensional environment, tracks objects including the person and obstacles, and records information. The monitoring safety application 120 monitors the person using the one or more cameras 103 and the one or more microphones 105. As an example, the monitoring safety application 120 records video, photographs, and/or audio. The drone 102 may determine whether the mobile computing device 106, the wearable device 104, and/or the person are currently moving or stationary. If the mobile computing device 106, the wearable device 104, and/or the person is moving, the drone 102 tracks and follows the person and continues to record video, photographs, and/or audio. In one embodiment, the monitoring safety application 120 streams the video and/or the audio to the alarm response server 108 and/or the call center server 112. In another embodiment, the monitoring safety application 120 stores the video, photographs, and/or the audio in the memory 118. The monitoring safety application 120 communicates data and messages with the mobile computing device 106, the alarm response server 108, and/or the call center server 112 using the communication network 114.

The drone 102 may further include an optional display/output device 107 and an input device 109. The display/output device 107 is used to provide status information about the drone 102 including a current battery level or fuel level, a flying status (e.g., ascending/descending), and other information. The output device 107 may be one or more light emitting diodes, e.g., a light emitting diode that flashes while the drone 102 is in operation. The display may indicate the status information. The display can be a liquid-crystal display, a light-emitting diode display, an organic light-emitting diode display, a touch screen display, an e-ink display, an e-paper display, and other displays. The input device 109 is used to interact with the drone 102 and may include one or more hardware buttons. The hardware buttons may include an on/off button and other buttons. The input device 109 may be included within the display if the display is a touch screen display. The input device 109 allows a user of the drone 102 to manipulate and interact with the monitoring safety application 120.

The drone 102 may also include an optional remote control receiver that operates with the input device 109 for receiving information from an optional remote control transmitter. The remote control transmitter transmits information to the remote control receiver to monitor, control, and operate the drone 102. The remote control transmitter may be a dedicated device comprising one or more processors and memory or a computer such as the alarm response server 108 or the call center server 112.

In one exemplary embodiment, a first drone 102 may communicate with a second drone 102. In one example, the first drone 102 and the second drone 102 may travel to the location and cooperate to simultaneously monitor video, photographs, and/or the audio from multiple vantage points and/or multiple angles. The first drone 102 and the second drone 102 may stream the video and/or the audio to the alarm response server 108 and/or the call center server 112. In addition, the first drone 102 and the second drone 102 may store the video, photographs, and/or the audio in the memory 118. The first drone 102 and the second drone 102 may communicate data and messages with the mobile computing device 106, the alarm response server 108, and/or the call center server 112 using the communication network 114.

In a second example, the first drone 102 may travel to the location at a first time and monitor video, photographs, and/or the audio. When the first drone 102 determines that the battery level reaches a particular level, the first drone 102 may send a message to the alarm response server 108 and/or a second drone 102. The second drone 102 may travel to the location at a second time and monitor video, photographs, and/or the audio. The first drone 102 and the second drone 102 may cooperate to seamlessly monitor video, photographs, and/or the audio for an extended period of time that may be longer than the life of a battery of a single drone. The second drone 102 may send a message to the alarm response server 108 and/or a third drone 102 and the monitoring process may continue by the third drone 102, and so on.

FIG. 1 illustrates a block diagram of the optional wearable device 104 according to an example embodiment. The wearable device 104 may be a computer having one or more processors 122 and memory 124, including but not limited to a watch, a necklace, a pendant, a hair clip, a hair tie, a pin, a tie clip/tack, a ring, a cufflink, a belt clip, a scarf, a pashmina, a wrap, a shawl, a garment, a keychain, another small mobile computing device, or a dedicated electronic device having a processor 122 and memory 124. The wearable device 104 may be a Bluetooth Low Energy (BLE, Bluetooth LE, Bluetooth Smart) Device based on the Bluetooth 4.0 specification or another specification. According to an example embodiment, the wearable device 104 and the mobile computing device 106 are paired and communicate wirelessly using a short-range wireless network, e.g., Bluetooth.

In another example, the wearable device 104 may create a personal area network and/or a mesh network for communicating with the one or more mobile computing devices 106 and/or the one or more drones 102. Additionally, the wearable device 104, the mobile computing device 106, and the one or more drones 102 may communicate using Zigbee, Wi-Fi, near field magnetic inductance, sonic (sound) waves, and/or infrared (light) waves. According to an example embodiment, the wearable device 104 may be a smart watch such as a GARMIN™ smart watch, a Pebble™ smart watch, a SAMSUNG™ Galaxy Gear smart watch, an ANDROID™ based smart watch, an APPLE™ and/or iOS™-based smart watch, a Tizen™ smart watch, and a VALRT™ wearable device, among others.

The one or more processors 122 may process machine/computer-readable executable instructions and data, and the memory 124 may store machine/computer-readable executable instructions and data including one or more applications, including a wearable safety application 126. The processor 122 and memory 124 are hardware. The memory 124 includes random access memory (RAM) and non-transitory memory, e.g., a non-transitory computer-readable medium such as one or more flash disks or hard drives. The non-transitory memory may include any tangible computer-readable medium including, for example, magnetic and/or optical disks, flash drives, and the like.

The wearable safety application 126 may be a component of an application and/or service executable by the wearable device 104. For example, the wearable safety application 126 may be a single unit of deployable executable code. The wearable safety application 126 may also be one application and/or a suite of applications for triggering an alarm notification. In one embodiment, the wearable safety application 126 sends an alarm notification directly to the alarm response server 108. In another embodiment, the wearable safety application 126 sends the alarm notification to the mobile computing device 106 and the mobile computing device 106 forwards the alarm notification to the alarm response server 108. The wearable safety application 126 may be a web-based application viewed in a browser on the wearable device 104 and/or a native application executed by the wearable device 104. The wearable safety application 126 may be downloaded from the Internet and/or digital distribution platforms, e.g., directly from a website or an app store such as the Pebble™ appstore, the (iOS™) App Store, and GOOGLE PLAY,™ among others. The wearable safety application 126 communicates messages with the mobile computing device 106 and/or the alarm response server 108 using the communication network 114.

The wearable device 104 may further include an optional display and an input device. The display is used to display visual components of the wearable safety application 126, such as at a user interface. In one example, the user interface may display a user interface of the wearable safety application 126. The display can be a liquid-crystal display, a light-emitting diode display, an organic light-emitting diode display, a touch screen display, an e-ink display, an e-paper display, and other displays. The input device is used to interact with the wearable safety application 126 and may include one or more hardware buttons. The input device may be included within the display if the display is a touch screen display. The input device allows a user of the wearable device 104 to manipulate and interact with the user interface of the wearable safety application 126.

FIG. 1 also illustrates a block diagram of the mobile computing device 106 according to an example embodiment. The mobile computing device 106 may be a computer having one or more processors 128 and memory 130, including but not limited to a server, laptop, desktop, tablet computer, smartphone, or a dedicated electronic device having a processor 128 and memory 130. The one or more processors 128 may process machine/computer-readable executable instructions and data, and the memory 130 may store machine/computer-readable executable instructions and data including one or more applications, including a mobile safety application 132. The processor 128 and memory 130 are hardware. The memory 130 includes random access memory (RAM) and non-transitory memory, e.g., a non-transitory computer-readable medium such as one or more flash disks or hard drives. The non-transitory memory may include any tangible computer-readable medium including, for example, magnetic and/or optical disks, flash drives, and the like.

The mobile safety application 132 may be a component of an application and/or service executable by the mobile computing device 106. For example, the mobile safety application 132 may be a single unit of deployable executable code. The mobile safety application 132 may also be one application and/or a suite of applications for triggering an alarm notification. In one embodiment, the mobile safety application 132 sends an alarm notification directly to the alarm response server 108. In another embodiment, the mobile safety application 132 receives the alarm notification from the wearable device 104 and the mobile computing device 106 forwards the alarm notification to the alarm response server 108. The mobile safety application 132 may be a web-based application viewed in a browser on the mobile computing device 106 and/or a native application executed by the mobile computing device 106. The application may be downloaded from the Internet and/or digital distribution platforms, e.g., directly from a website, the Mac™ App Store, the (iOS™) App Store, and/or GOOGLE PLAY™, among others. According to an example embodiment, the mobile safety application 132 is an iOS™ application, an Android™ application, or a Windows™ Phone application. The mobile safety application 132 communicates messages with the drone 102, the wearable device 104 and/or the alarm response server 108 using the communication network 114.

The mobile computing device 106 includes global positioning system (GPS) hardware. The GPS hardware communicates with a GPS satellite-based positioning system. The mobile computing device 106 may further include an optional display and an input device. The display is used to display visual components of the mobile safety application 132, such as at a user interface. In one example, the user interface may display a user interface of the mobile safety application 132. The display can be a liquid-crystal display, a light-emitting diode display, an organic light-emitting diode display, a touch screen display, an e-ink display, an e-paper display, and other displays. The input device is used to interact with the mobile safety application 132 and may include a mouse, a keyboard, a trackpad, and/or the like. The input device may be included within the display if the display is a touch screen display. The input device allows a user of the mobile computing device 106 to manipulate and interact with the user interface of the mobile safety application 132.

FIG. 1 further illustrates a block diagram of the alarm response server 108 according to an example embodiment. According to an aspect of the present disclosure, the alarm response server 108 is a computer having one or more processors 134 and memory 136. The alarm response server 108 may be, for example, a laptop, desktop, a server, tablet computer, mobile computing device (e.g., a smart phone) or a dedicated electronic device having a processor 134 and memory 136. The alarm response server 108 includes one or more processors 134 to process data and memory 136 to store machine/computer-readable executable instructions and data including an alarm response application 138. The processor 134 and memory 136 are hardware. The memory 136 includes non-transitory memory, e.g., random access memory (RAM) and one or more hard disks. The non-transitory memory may include any tangible computer-readable medium including, for example, magnetic and/or optical disks, flash drives, and the like. The data associated with the alarm response application 138 may be stored in a structured query language (SQL) server database or another appropriate database management system within memory 136 and/or in the one or more databases 110. Additionally, the memory 136 and/or the databases 110 may also include a dedicated file server having one or more dedicated processors, random access memory (RAM), a Redundant Array of Inexpensive Disks (RAID) hard drive configuration, an Ethernet interface or other communication interface, and a server-based operating system.

The alarm response server 108 may further include an optional display and an input device. The display is used to display visual components of the alarm response application 138, such as at a user interface. In one example, the user interface may display a user interface of the alarm response application 138. The display can be a liquid-crystal display, a light-emitting diode display, an organic light-emitting diode display, a touch screen display, an e-ink display, an e-paper display, and other displays. The input device is used to interact with the alarm response application 138 and may include a mouse, a keyboard, a trackpad, and/or the like. The input device may be included within the display if the display is a touch screen display. The input device allows a user of the alarm response server 108 to manipulate and interact with the user interface of the alarm response application 138.

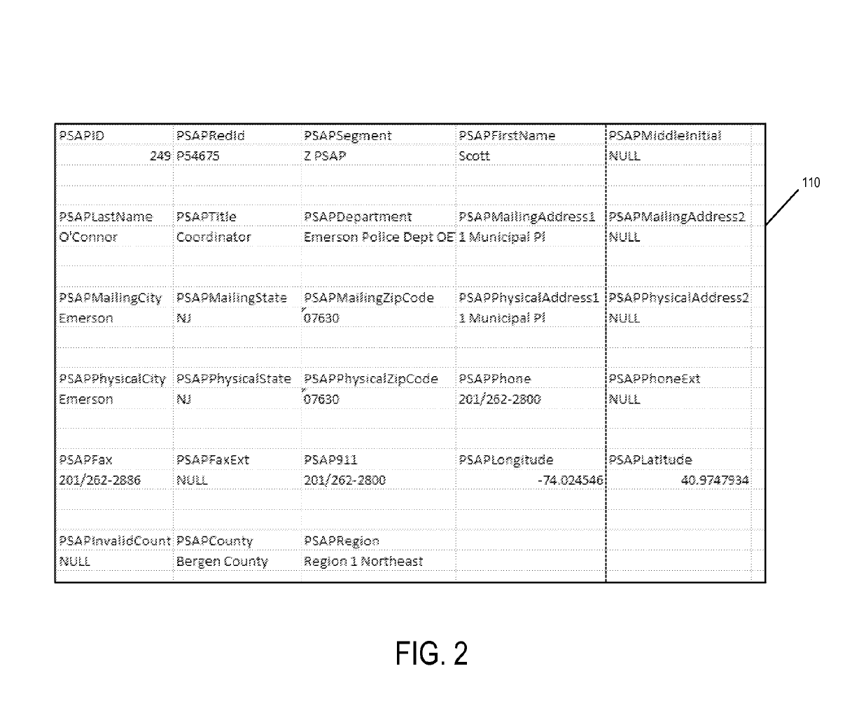

According to an example embodiment, the one or more databases 110 may store user information associated with one or more users of the wearable safety application 126 and/or the mobile safety application 132 such as identifying information. In addition, the one or more databases 110 may store alarm notification information including a record of each alarm notification received by the alarm response server 108. Each record may include a unique alarm notification identifier and the unique identifier associated with corresponding identifying information. The record also may include location information and other information. In addition, the one or more databases 110 may store PSAP information as shown in FIG. 2.

FIG. 1 illustrates a block diagram of the call center server 112 according to an example embodiment. The call center server 112 may be associated with a PSAP, e.g., a 911 emergency dispatch center. According to an aspect of the present disclosure, the call center server 112 is a computer having one or more processors 140 and memory 142. The call center server 112 may be, for example, a laptop, desktop, a server, tablet computer, mobile computing device (e.g., a smart phone) or a dedicated electronic device having a processor 140 and memory 142. The call center server 112 includes one or more processors 140 to process data and memory 142 to store machine/computer-readable executable instructions and data including an emergency dispatch application 144. The processor 140 and memory 142 are hardware. The memory 142 includes non-transitory memory, e.g., random access memory (RAM) and one or more hard disks. The non-transitory memory may include any tangible computer-readable medium including, for example, magnetic and/or optical disks, flash drives, and the like. The data associated with the emergency dispatch application 144 may be stored in a structured query language (SQL) server database or another appropriate database management system within memory 142 and/or in one or more databases associated with the call center server 112. Additionally, the memory 142 and/or the databases associated with the call center server 112 may also include a dedicated file server having one or more dedicated processors, random access memory (RAM), a Redundant Array of Inexpensive Disks (RAID) hard drive configuration, an Ethernet interface or other communication interface, and a server-based operating system.

The call center server 112 may further include an optional display and an input device. The display is used to display visual components of the emergency dispatch application 144, such as at a user interface. In one example, the user interface may display a user interface of the emergency dispatch application 144. The display can be a liquid-crystal display, a light-emitting diode display, an organic light-emitting diode display, a touch screen display, an e-ink display, an e-paper display, and other displays. The input device is used to interact with the emergency dispatch application 144 and may include a mouse, a keyboard, a trackpad, and/or the like. The input device may be included within the display if the display is a touch screen display. The input device allows a user of the call center server 112 to manipulate and interact with the user interface of the emergency dispatch application 144.

In one embodiment, a user may configure the wearable device 104 and/or the mobile computing device 106. The user may download and/or install the wearable safety application 126 in memory 124 on the wearable device 104 and the mobile safety application 132 in memory 130 on the mobile computing device 106. In an example, the user downloads and installs the wearable safety application 126 on a Pebble™ wearable device and the user downloads and installs the mobile safety application 132 on an iOS™-based smart phone. Once installed, the user may configure the wearable safety application 126 and the mobile safety application 132 for use. Using the user interface of the mobile safety application 132, the user interface of the wearable safety application 126, or another interface (e.g., a web-based interface), the user may enter setup and/or configuration information comprising identifying information. The identifying information may include one or more of a name (first and last), one or more email addresses, one or more telephone numbers including a telephone number of the mobile computing device 106 or the wearable device 104, one or more addresses, a height, a weight, an eye color, a hair color, a gender, a photograph, an alarm code for disabling an alarm notification, and a secret code for discreetly indicating that the user is in immediate need of assistance, among other information. As an example, the secret code may be automatically derived from the alarm code. If the alarm code is entered as 1234, the secret code may be automatically set by the mobile safety application 132 as 1235. In addition, the user may provide information associated with one or more lifelines, e.g., a person to contact in the event of an emergency. The information associated with the one or more lifelines may include a name, one or more email addresses, and one or more telephone numbers, among other information.

The wearable device 104, the mobile computing device 106, or another computer sends the identifying information to the alarm response server 108 via the communication network 114. The alarm response server 108 receives the identifying information and stores the identifying information in the memory 136 and/or the database 110. The alarm response server 108 associates the identifying information with a unique identifier (e.g., a member identifier) and transmits the unique identifier to the wearable device 104 and/or the mobile computing device 106. The wearable safety application 126 and/or the mobile safety application 132 receive the unique identifier and store the unique identifier in memory 124 and/or memory 130. At this point, the wearable safety application 124 and the mobile safety application 132 are configured and ready for use.

According to an example embodiment, in the event of an emergency, the user may trigger an alarm notification representing an instant emergency alarm that deploys the drone 102 and notifies first responders (e.g., a 911 PSAP) using the wearable device 104 and/or the mobile computing device 106.

After the mobile safety application 132 is configured and ready for use, the mobile safety application 132 may operate in one of two exemplary operation modes. In a first monitoring mode, the mobile safety application 132 continually determines whether the user is touching the touchscreen of the mobile computing device 106. In one example, the user may keep a finger on the touchscreen while the mobile computing device 106 is located in a pocket. In another example, the user may keep a finger on the touchscreen while holding the mobile computing device 106 as if the mobile computing device 106 is being used to place a telephone call. If the user stops touching the touchscreen of the mobile computing device 106, an alarm notification may be triggered. This may occur if the user is attacked and/or the user drops the mobile computing device 106. The alarm notification also may be triggered if the user enters the secret passcode. A countdown may begin after the alarm notification is triggered. During this countdown, the user may stop the countdown or disarm the mobile safety application 132. However, if the user does not stop the countdown or disarm the mobile safety application 132, the alarm notification is confirmed.

In a second monitoring mode, the mobile safety application 132 may automatically trigger an alarm notification after a particular preset period of time, e.g., ten minutes. While in the second monitoring mode, the mobile safety application 132 may display a timer that indicates how much of the particular period of time is left until the alarm notification is triggered. As an example, it may take the user approximately six minutes to travel from their car or a train station to their apartment. The user may desire to use the second monitoring mode of the mobile safety application 132 while traveling from their car or the train station to their apartment. The user may disarm the mobile safety application 132 upon arrival at the apartment. However, after the particular period of time ends, the alarm notification is triggered. The alarm notification also may be triggered if the user enters the secret passcode. A countdown may begin after the alarm notification is triggered. During this countdown, the user may stop the countdown or disarm the mobile safety application 132. However, if the user does not stop the countdown or disarm the mobile safety application 132, the alarm notification is confirmed.

The wearable safety application 126 also may trigger the alarm notification. In one example, the user may press and hold two hardware buttons on the wearable device 104 for a particular period of time, e.g., four seconds. In another example, the user may press and hold one hardware button on the wearable device 104 for the particular period of time. In a further example, the user may press a hardware button on the wearable device 104 a particular number of times consecutively in a particular period of time, e.g., three to ten times in twenty seconds. In another example, the user may press the touch screen of the wearable device 104 a particular number of times consecutively in a particular period of time.

The wearable device 104 may include a radio frequency (RF) transceiver or another transceiver for transmitting the alarm notification to the mobile computing device 106 and/or the alarm response server 108. The wearable device 104 may include a microphone for receiving a voice activated alarm notification, an accelerometer for detecting an acceleration greater than a particular threshold to generate an alarm notification (e.g., a hard fall), a gyroscope for detecting rotation greater than a particular threshold to generate an alarm notification (e.g., a hard fall), and a biometric device to receive an alarm notification. In one aspect, the biometric device may be a fingerprint recognition device to determine unique patterns in one or more fingers of the user or a retina scanner to determine unique patterns associated with a retina of the user. In another aspect, the biometric device may be a heart rate monitor to measure and/or record a heart rate of a user. The biometric device also may detect a heart attack and/or an abnormal heart rate. The biometric device may store information associated with the heart rate in memory 124 and memory 130 to provide historic contextual data for a normal and an abnormal heart rate. If the heart rate is lower than a particular threshold or higher than a particular threshold, the heart rate monitor may detect distressed health conditions, a heart attack and/or conditions indicative of a heart attack and generate an alarm notification that may be sent to one or more PSAPs and first responders. Of course, this is just one example of user health monitoring that may be executed using the systems and methods taught herein. There are numerous monitored conditions that may be used to generate an alarm notification, including temperature, breathing rate, etc.

After the wearable device 104 triggers the alarm, the wearable device 104 sends an alarm notification message to the mobile computing device 106. The alarm notification message may be sent by the wearable device 104 using a Bluetooth network or another short-range wireless network. The mobile computing device 106 reverse geocodes a current location of the mobile computing device 106 using the global positioning system (GPS) hardware. The GPS hardware communicates with a GPS satellite-based positioning system. The GPS hardware may be an assisted GPS system, e.g., A-GPS or aGPS, or may be a standalone GPS. Standalone GPS only uses radio signals from the satellite-based positioning system. An assisted GPS system uses network resources available to the mobile computing device 106 and/or the wearable device 104 to locate and use the satellite-based positioning system in poor signal conditions, such as in a city where signals bounce off of buildings or pass through walls or tree cover.

The mobile computing device 106 sends or forwards the alarm notification message with the current location information and the unique identifier to the alarm response server 108 via the communication network 112. The alarm response server 108 receives the alarm notification message, transmits a unique alarm identifier to the mobile computing device 106 that corresponds with this particular alarm notification, and determines one or more PSAPs based on the current location information. In one example, the alarm response application 138 of the alarm response server 108 determines three PSAPs that are closest to the current location of the mobile computing device 106 by querying the one or more databases 110 using the current location information, e.g., a latitude value and a longitude value. In another example, the alarm response application 138 of the alarm response server 108 determines three PSAPs that have a highest safety score. The safety score may be based on the current location of the mobile computing device 106, a historical response time of the PSAP, a PSAP service rating (e.g., one to five stars), and other service-level agreement based factors.

The alarm response application 138 may generate a user interface on the display of the alarm response server 108. The user interface may include information associated with the one or more PSAPs, the identifying information, a map showing the current location of the user, and the monitoring information from the drone 102, among other information. The user interface may include a button or other user interface element for indicating that the alarm notification is a false alarm, one or more buttons or other user interface elements to control and monitor the one or more drones 102, and another button or other user interface element for forwarding the alarm notification to the call center server 112.

After or concurrently with the determination of the one or more PSAPs, the alarm response application 138 determines a telephone number and/or email address in the one or more databases 110 associated with the unique identifier. The alarm response application 138 of the alarm response server 108 initiates one or more automated telephone calls, sends an email, and/or sends a text message (SMS/MMS) to the mobile computing device 106 or the wearable device 104 to verify a condition of the instant emergency alarm.

The user of the wearable device 104 and/or the mobile computing device 106 may indicate that the instant emergency alarm was a false alarm by providing the alarm passcode, e.g., one or more numbers such as 1234. The alarm passcode may be provided to a human call representative associated with the alarm response server 108. In another instance, the text message and the email may include a uniform resource locator (URL) to direct the user to a web page having a form to receive the alarm passcode. The user of the mobile computing device 106 may view the web page and transmit the alarm passcode to the alarm response server 108. Using the database 110, the alarm response server 108 confirms that the alarm passcode is correct, e.g., this is a false alarm, and the process may end.

The user of the wearable device 104 and/or the mobile computing device 106 may indicate that the instant emergency alarm was not a false alarm by providing the secret passcode, e.g., one or more numbers such as 911 or 1235. The secret passcode may be provided to the human call representative associated with the alarm response server 108. In another instance, the text message and the email may include the URL that directs the user to the web page having the form to receive the secret passcode. The user of the mobile computing device 106 may view the web page and transmit the secret passcode to the alarm response server 108. Using the database 110, the alarm response server 108 confirms that the secret passcode is correct or not correct. If the secret passcode is correct, the alarm response server 108 sends the alarm notification with the identifying information and the location information to the emergency dispatch application 138 of the call center server 112 via the communication network. Optionally, the alarm response server 108 sends the alarm notification with the identifying information and the location information to the one or more lifelines by initiating an automated telephone call, sending an email, and/or sending a text message (SMS/MMS) to the one or more lifelines. The email and text message may include a URL that provides detailed information about the alarm notification including a map showing the current location of the user.

If the alarm response server 108 does not receive the correct alarm passcode after a particular period of time (e.g., one minute), the alarm response server 108 sends the alarm notification with the identifying information and the location information to the emergency dispatch application 144 of the call center server 112 via the communication network. Optionally, the alarm response server 108 sends the alarm notification with the identifying information and the location information to the one or more lifelines by initiating an automated telephone call, sending an email, and/or sending a text message (SMS/MMS) to the one or more lifelines. The email and text message may include a URL that provides detailed information about the alarm notification including a map showing the current location of the user.

If the alarm response server 108 does not receive the correct alarm passcode after the particular period of time, the alarm response server 108 sends the identifying information and the current location information to the drone 102. The drone 102 receives the identifying information and the current location information and stores the identifying information and the current location information in the memory 118. The drone 102 determines the quickest and/or shortest route to the current location using the current location information, weather conditions, and obstacles. The drone 102 travels to the current location using the route and upon arrival begins monitoring activity at the current location. As an example, the drone 102 hovers at a particular altitude and records video, photographs, and/or audio using the one or more cameras and the one or more microphones. The drone 102 may stream and/or transmit the video, photographs, and/or the audio to the alarm response server 108 and/or the call center server 112. If the person, the mobile computing device 106, and/or the wearable device 104 begins moving while the drone 102 is monitoring, the drone 102 tracks and follows the person and continues to record video, photographs, and/or audio.

In one embodiment, the drone 102 continues to record and/or stream the at least one of video, audio, and photographic information for a particular period of time, e.g., ten minutes, or until the drone battery level reaches a critical level. The critical level may be based upon a distance that the drone 102 is from the hangar. Upon reaching the critical level, the drone 102 stops recording and/or streaming to have sufficient battery power to return to the hangar. In another embodiment, the drone 102 continues to record and/or stream the at least one of video, audio, and photographic information until the drone 102 receives a message from one of the alarm response server 108 and/or the call center server 112 to stop recording. After the drone 102 stops recording, the drone 102 follows a reverse route or another route back to its hangar. The reverse route may be a route that is opposite of the route that the drone used to reach the location. In one aspect, upon arrival at the hangar and/or connecting to the communications network 114, the drone 102 transmits the at least one of video, audio, and photographic information to the alarm response server 108 and/or the call center server 112. The video, audio, and photographic information may be stored in the database 110 and associated with the unique identifier and the unique alarm identifier.

According to an example embodiment, the one or more drones 102, the one or more wearable devices 104, the one or more mobile computing devices 106, the one or more alarm response servers 108, the one or more databases 110, and the one or more call center servers 112 communicate using a web application programming interface (API) comprising a defined request/response message system. According to one aspect, the message system is based on Javascript Object Notation (JSON) and the web API is a RESTful web API based on Representational State Transfer (REST).

The web API includes one or more HTTP methods including alert activation, alert cancel, alert triggered, alert silent alarm, and alert location update, among other methods.

Alert activation may be called when the user activates one of the monitoring mode and the timer mode. When the alert activation is called, a record is created in the database 110 having a unique alert/alarm identifier. As an example, the alert activation uniform resource locator (URL) comprises http://a.llr1.com/rest/AlertActivation. The alert activation input parameters include an alert latitude, an alert longitude, a member ID (unique identifier), an alert type (monitoring or timer), and an alarm minutes value. The alarm minutes value is associated with the second timer mode. The alert activation output parameters include a status code, a status description, and an alert ID (e.g., a unique alert/alarm identifier that represents this particular alert notification). The unique alert identifier may be used to reference a particular alarm notification, e.g., 27307.

Sample alert activation header & body:

Authorization: OAuth

Content-Type: application/json\r\n\r\n\r\n

{“AlertLatitud”:“41.903507”,“AlertLogitud”:“−87.987227”,“MemberId”:“1”,“AlertType”:“M”,“AlarmMinutes”:“0”}\r\n

LIVE Response Successful:

### {“StatusCode”:0,“StatusDescription”:“Success.”,“AlertId”:“27307”}

LIVE Response Failed: {“StatusCode”:1,“StatusDescription”:“No matching ‘Member’.”,“AlertId”:“0”}

Alert cancel may be called when the user correctly enters the alarm passcode to deactivate the alert. Alert cancel is applicable to both the monitoring mode and the timer mode. As an example, the alert cancel URL comprises http://a.llr1.com/rest/AlertCancel. The alert cancel input parameters include a unique alert identifier. The alert cancel output parameters include a status code and a status description.

Sample alert cancel header & body:

Authorization: OAuth

Content-Type: application/json\r\n\r\n\r\n

{“AlertId”:“27307”}\r\n

LIVE Response Successful: {“StatusCode”:0,“StatusDescription”:“Success.”}

LIVE Response Failed: {“StatusCode”:1,“StatusDescription”:“Invalid Alert Id—Alert Id not found”}

Alert trigger may be called when the user is in monitoring mode and the user ends monitoring mode. Monitoring mode may end when the user removes a finger from a touchscreen of the mobile computing device 106. As an example, the alert trigger URL comprises http://a.llr1.com/rest/AlertTrigger. The alert trigger input parameters include a unique alert identifier, an alert latitude, and an alert longitude. The alert trigger output parameters include a status code and a status description.

Sample alert trigger header & body:

Authorization: OAuth

Content-Type: application/json\r\n\r\n\r\n

{“AlertId”:“167”,“AlertLatitud”: “45.903507”,“AlertLogitud”:“−82.987227”}\r\n

LIVE Response Successful: {“StatusCode”:0,“StatusDescription”:“Success.”}

LIVE Response Failed: {“StatusCode”:1,“StatusDescription”:“Invalid Alert Id—Alert Id not found”}

Alert silent alarm may be called when the user is in monitoring mode and the user enters the secret password to trigger the alarm. As an example, the alert silent alarm URL comprises http://a.llr1.com/rest/AlertSilentAlarm. The alert silent alarm input parameters include a unique alert identifier. The alert silent alarm output parameters include a status code and a status description.

Sample alert silent alarm header & body:

Authorization: OAuth

Content-Type: application/json\r\n\r\n\r\n

{“AlertId”:“4”}\r\n

LIVE Response Successful: {“StatusCode”:0,“StatusDescription”:“Success.”}

LIVE Response Failed: {“StatusCode”:1,“StatusDescription”:“Invalid Alert Id—Alert Id not found”}

Alert location update may be called to update location information associated with a particular alarm notification. As an example, the alert location update may be called at a particular interval of time after the alarm notification, e.g., every ten seconds. In another example, the alert location update may be called when the mobile computing device 106 and/or the wearable device 104 moves a particular distance, e.g., every 37 feet of movement. Based on the alert location update, the drone 102, the alarm response server 108, and the call center server 112 may determine how fast the mobile computing device 106 and/or the wearable device 104 are moving by evaluating the difference between each alert location update. The drone 102, the alarm response server 108, and the call center server 112 may determine an instantaneous speed of the mobile computing device 106 and/or the wearable device 104 based on the distance traveled with respect to time. As an example, the alert location update URL comprises http://a.llr1.com/rest/AlertLocationUpdate. The alert location update input parameters include a unique alert identifier, an alert location latitude, and an alert location longitude. The alert location update output parameters include a status code and a status description.

Sample alert location update header & body:

Authorization: OAuth

Content-Type: application/json\r\n\r\n\r\n

{“AlertId”:“27307”,“AlertLocationLatitud”:“41.90350”,“AlertLocationLogitud”:“−87.987227”}\r\n

LIVE Response: {“StatusCode”:0,“StatusDescription”:“Success.”}

FIG. 2 illustrates example information in the alarm response database 110 according to an example embodiment. According to an example embodiment, the alarm response database may store PSAP information. Each PSAP in the United States and throughout the world may have database fields/attributes stored in the alarm response database 110. As shown in FIG. 2, the database fields/attributes may include one or more of a PSAP ID, a PSAP RedID, a PSAP Segment, a PSAP First Name, a PSAP Middle Initial, a PSAP Last Name, a PSAP Department, a PSAP Mailing Address (1), a PSAP Mailing Address (2), a PSAP Mailing City, a PSAP Mailing State, a PSAP Mailing Zip Code, a PSAP Physical Address (1), a PSAP Physical Address (2), a PSAP Physical City, a PSAP Physical State, a PSAP Physical Zip Code, a PSAP Phone Number, a PSAP Phone Extension, a PSAP Fax Number, a PSAP Fax Extension, a PSAP911 Phone Number, a PSAP Longitude, a PSAP Latitude, a PSAP InvalidCount, a PSAP County, and a PSAP Region, among others.

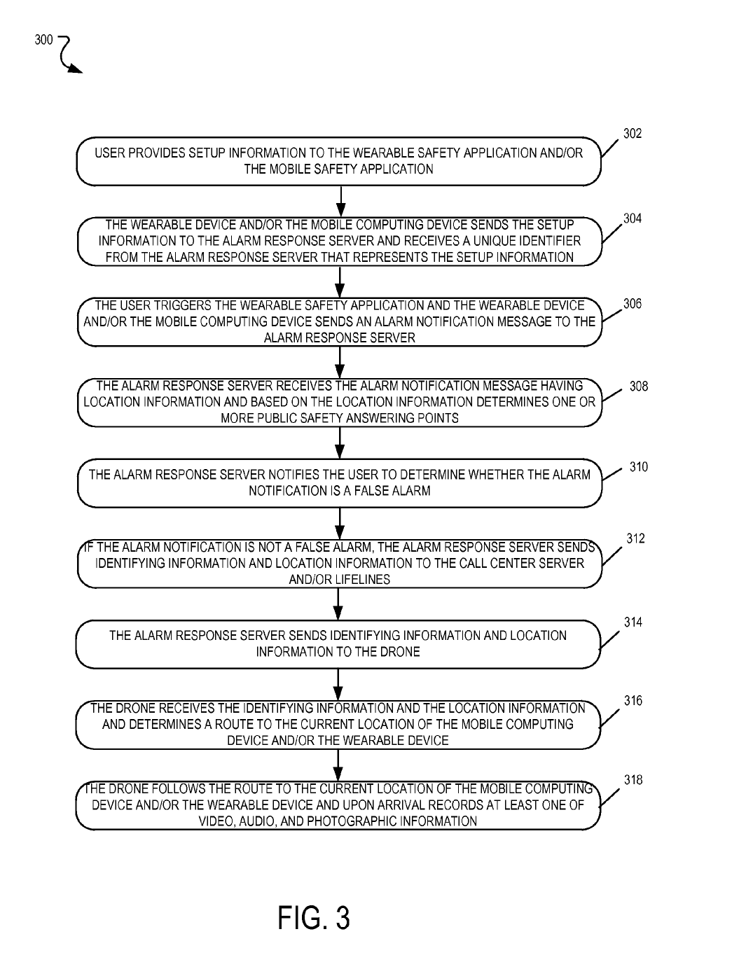

FIG. 3 illustrates a flowchart of a process for triggering an alarm notification and monitoring by the drone 102, according to an example embodiment. The process 300 shown in FIG. 3 begins in step 302. In step 302, the user of the wearable device 104 and/or the mobile computing device 106 provides setup information to the wearable safety application 126 and/or the mobile safety application 132. The setup information comprises the identifying information. In step 304, the wearable safety application 126 of the wearable device 104 and/or the mobile safety application 132 of the mobile computing device 106 send the setup information including the identifying information to the alarm response server 108 via the communication network 114. The alarm response server 108 stores the identifying information in the one or more databases 110 and sends a unique identifier that represents the identifying information to the wearable device 104 and/or the mobile computing device 106. The wearable device 104 and/or the mobile computing device 106 receive the unique identifier and store the unique identifier in memory 124 and/or memory 130.

In step 306, in the event of an emergency, the user triggers the wearable device 104 and/or the mobile computing device 106. In one embodiment, the wearable safety application 124 receives the trigger and sends an alarm notification message to the mobile computing device 106 via Bluetooth or another short-range wireless protocol. In an additional embodiment, the mobile safety application 132 receives the trigger via the monitoring mode or the timer mode. The mobile computing device 106 reverse geocodes a current location of the mobile computing device 106. In another embodiment, the wearable device 104 reverse geocodes a current location of the wearable device 104 and provides this current location with the alarm notification message. The mobile computing device 106 sends the alarm notification message including current location information and the unique identifier to the alarm response server 108.

In step 308, the alarm response server 108 receives the alarm notification message having the current location information and based on the current location information and the PSAP information in the database 110 determines one or more PSAPs. In response to the alarm notification message, the alarm response server 108 may send the mobile computing device 106 and/or the wearable device 104 a unique alarm identifier that represents the alarm notification.

In step 310, the alarm response server 108 notifies the user to determine whether the alarm notification is a false alarm. The alarm response server 108 may send one or more of a telephone call, an email, and a message to the mobile computing device 106 and/or the wearable device 104. If the user provides a correct alarm code, the process may end. However, if the alarm notification is not a false alarm and if the user does not provide a correct alarm code or provides a secret code, in step 312, the alarm response server 108 sends the alarm notification message including the identifying information and the current location information to the call center server 112. In addition, the alarm response server 108 may send the identifying information and the current location information to the one or more lifelines.

In step 314, the alarm response server 108 sends the identifying information and the current location information to the drone 102. In step 316, the drone 102 receives the identifying information and the current location information and stores the identifying information and the current location information in the memory 118. The drone 102 determines a shortest and/or quickest route from its hangar to the current location of the mobile computing device 106 and/or the wearable device 104. The route may be based on weather conditions and obstacles.

In step 318, the drone follows the route to the current location of the mobile computing device 106 and/or the wearable device 104. Upon arrival, the drone 102 records at least one of video, audio, and photographic information using the one or more cameras and the one or more microphones. In one embodiment, the drone 102 streams the at least one of video, audio, and photographic information to the alarm response server 108 and/or the call center server 112. In one embodiment, the drone 102 continues to record and/or stream the at least one of video, audio, and photographic information for a particular period of time, e.g., ten minutes, or until the drone battery level reaches a critical level. In another embodiment, the drone 102 continues to record and/or stream the at least one of video, audio, and photographic information until the drone 102 receives a message from the alarm response server 108, the call center server 112, a remote control, or another computing device to stop recording. After the drone 102 stops recording, the drone 102 follows a reverse route or another route back to its hangar. In one aspect, upon arrival at the hangar, the drone 102 transmits the at least one of video, audio, and photographic information to the alarm response server 108 and/or the call center server 112. The video, audio, and photographic information may be stored in the database 110 and associated with the unique identifier and/or the unique alarm identifier.

Although the embodiment described above indicates that the mobile computing device 106 sends the alarm notification message to the alarm response server 108, according to another embodiment, the wearable device 104 may directly send the alarm notification message to the alarm response server 108.

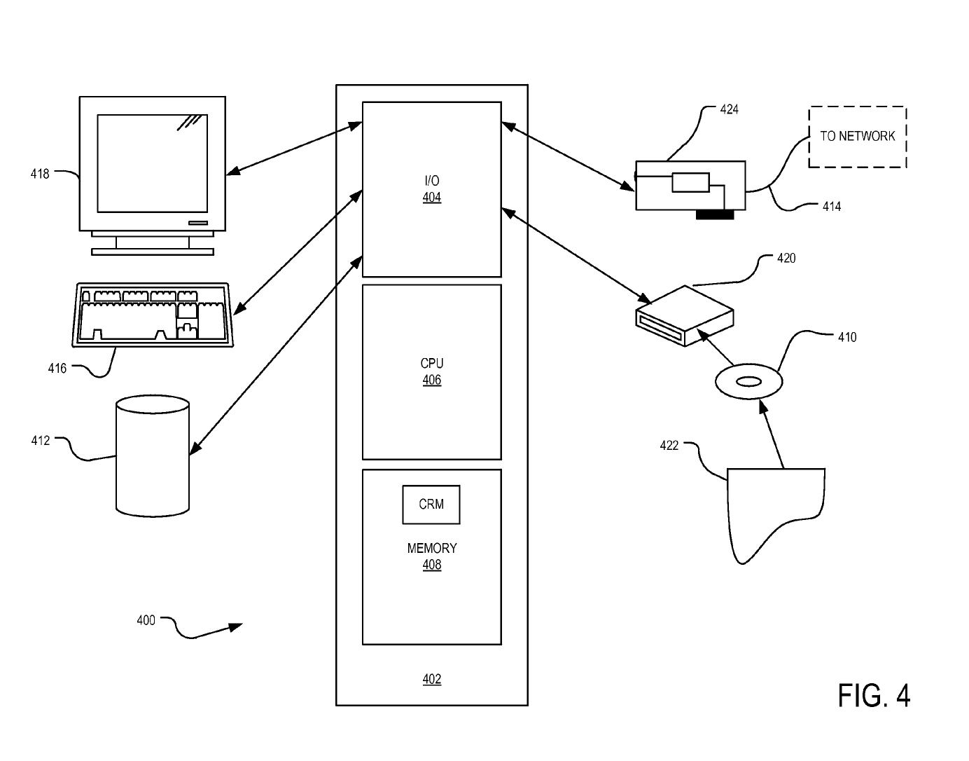

FIG. 4 illustrates an example computing system 400 that may implement portions of the various systems described herein, such as the drone 102, the wearable device 104, the mobile computing device 106, the alarm response server 108, the call center server 112, and methods discussed herein, such as process 300. A general-purpose computer system 400 is capable of executing a computer program product to execute a computer process. Data and program files may be input to the computer system 400, which reads the files and executes the programs therein such as the monitoring safety application 120, the wearable safety application 126, the mobile safety application 132, the alarm response application 138, and the emergency dispatch application 144. Some of the elements of a general-purpose computer system 400 are shown in FIG. 4 wherein a processor 402 is shown having an input/output (I/O) section 404, a central processing unit (CPU) 406, and a memory section 408. There may be one or more processors 402, such that the processor 402 of the computer system 400 comprises a single central-processing unit 406, or a plurality of processing units, commonly referred to as a parallel processing environment. The computer system 400 may be a conventional computer, a server, a distributed computer, or any other type of computer, such as one or more external computers made available via a cloud computing architecture. The presently described technology is optionally implemented in software devices loaded in memory 408, stored on a configured DVD/CD-ROM 410 or storage unit 412, and/or communicated via a wired or wireless network link 414, thereby transforming the computer system 400 in FIG. 4 to a special purpose machine for implementing the described operations.

The memory section 408 may be volatile media, nonvolatile media, removable media, non-removable media, and/or other media or mediums that can be accessed by a general purpose or special purpose computing device. For example, the memory section 408 may include non-transitory computer storage media and communication media. Non-transitory computer storage media further may include volatile, nonvolatile, removable, and/or non-removable media implemented in a method or technology for the storage (and retrieval) of information, such as computer/machine-readable/executable instructions, data and data structures, engines, program modules, and/or other data. Communication media may, for example, embody computer/machine-readable/executable, data structures, program modules, algorithms, and/or other data. The communication media may also include an information delivery technology. The communication media may include wired and/or wireless connections and technologies and be used to transmit and/or receive wired and/or wireless communications.

The I/O section 404 is connected to one or more user-interface devices (e.g., a keyboard 416 and a display unit 418), a disc storage unit 412, and a disc drive unit 420. Generally, the disc drive unit 420 is a DVD/CD-ROM drive unit capable of reading the DVD/CD-ROM medium 410, which typically contains programs and data 422. Computer program products containing mechanisms to effectuate the systems and methods in accordance with the presently described technology may reside in the memory section 404, on a disc storage unit 412, on the DVD/CD-ROM medium 410 of the computer system 400, or on external storage devices made available via a cloud computing architecture with such computer program products, including one or more database management products, web server products, application server products, and/or other additional software components. Alternatively, a disc drive unit 420 may be replaced or supplemented by a floppy drive unit, a tape drive unit, or other storage medium drive unit. The network adapter 424 is capable of connecting the computer system 400 to a network via the network link 414, through which the computer system can receive instructions and data. Examples of such systems include personal computers, Intel or PowerPC-based computing systems, AMD-based computing systems and other systems running a Windows-based, a UNIX-based, or other operating system. It should be understood that computing systems may also embody devices such as Personal Digital Assistants (PDAs), mobile phones, tablets or slates, multimedia consoles, gaming consoles, set top boxes, etc.

When used in a LAN-networking environment, the computer system 400 is connected (by wired connection and/or wirelessly) to a local network through the network interface or adapter 424, which is one type of communications device. When used in a WAN-networking environment, the computer system 400 typically includes a modem, a network adapter, or any other type of communications device for establishing communications over the wide area network. In a networked environment, program modules depicted relative to the computer system 400 or portions thereof, may be stored in a remote memory storage device. It is appreciated that the network connections shown are examples of communications devices for and other means of establishing a communications link between the computers may be used.

In an example implementation, source code executed by the drone 102, the wearable device 104, the mobile computing device 106, the alarm response server 108, and the call center server 112, a plurality of internal and external databases including the database 110, source databases, and/or cached data on servers are stored in memory 118 of the drone 102, memory 124 of the wearable device 104, memory 130 of the mobile computing device 106, memory 136 of the alarm response server 108, memory 142 of the call center server 112, or other storage systems, such as the disk storage unit 412 or the DVD/CD-ROM medium 410, and/or other external storage devices made available and accessible via a network architecture. The source code executed by the drone 102, the wearable device 104, the mobile computing device 106, the alarm response server 108, and the call center server 112 may be embodied by instructions stored on such storage systems and executed by the processor 402.

The processor 402, which is hardware, may perform some or all of the operations described herein. Further, local computing systems, remote data sources and/or services, and other associated logic represent firmware, hardware, and/or software configured to control operations of the drone safety alert monitoring system 100 and/or other components. Such services may be implemented using a general-purpose computer and specialized software (such as a server executing service software), a special purpose computing system and specialized software (such as a mobile device or network appliance executing service software), or other computing configurations. In addition, one or more functionalities disclosed herein may be generated by the processor 402 and a user may interact with a Graphical User Interface (GUI) using one or more user-interface devices (e.g., the keyboard 416, the display unit 418, and the user devices 404) with some of the data in use directly coming from online sources and data stores. The system set forth in FIG. 4 is but one possible example of a computer system that may employ or be configured in accordance with aspects of the present disclosure.

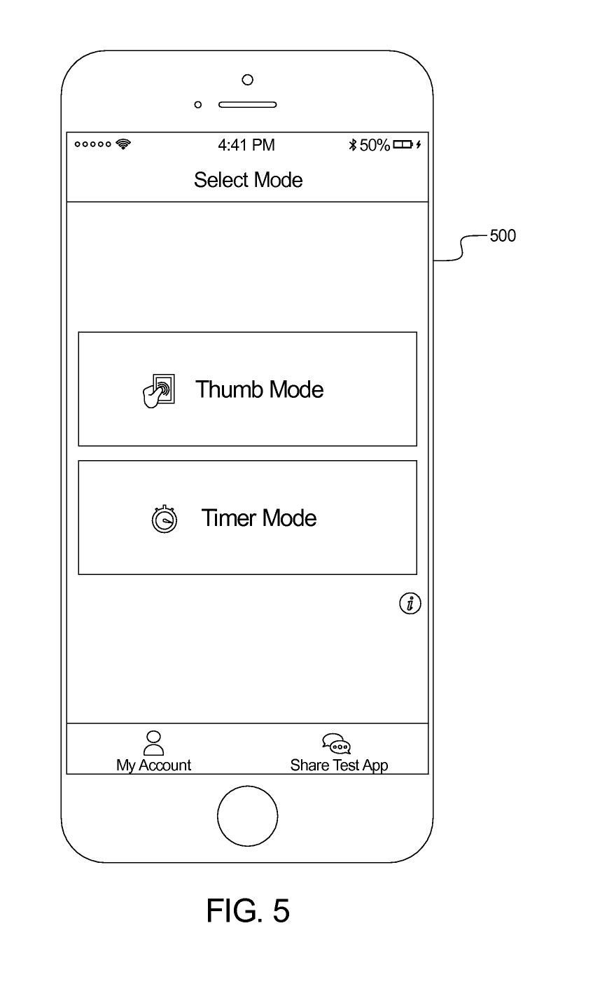

FIG. 5 illustrates an example screenshot 500 of the mobile safety application 132 executed by the mobile computing device 106 according to an example embodiment. As shown in FIG. 5, the mobile safety application 132 may operate in the first monitoring mode (e.g., thumb mode) or the second timer mode. If the user selects the thumb mode user interface button, the mobile safety application 132 enters the first monitoring mode. If the user selects the timer mode user interface button, the mobile safety application 132 enters the second timer mode.

FIG. 6 illustrates another example screenshot 600 of the mobile safety application 132 executed by the mobile computing device 106 according to an example embodiment. As shown in FIG. 6, the mobile safety application 132 is operating in the first monitoring mode. In the first monitoring mode, the mobile safety application 132 continually determines whether the user is touching the touchscreen of the mobile computing device 106. If the user stops touching the touchscreen of the mobile computing device 106, an alarm notification may be triggered. This may occur if the user is attacked and/or the user drops the mobile computing device 106. The alarm notification also may be triggered if the user enters the secret passcode. A countdown may begin after the alarm notification is triggered. During this countdown, the user may stop the countdown or disarm the mobile safety application 132. However, if the user does not stop the countdown or disarm the mobile safety application 132, the alarm notification is confirmed.

FIG. 7 illustrates another example screenshot 700 of the mobile safety application 132 executed by the mobile computing device 106 according to an example embodiment. As shown in FIG. 7, the mobile safety application 132 is operating in the second timer mode. As shown in the screenshot 700, the user interface of the mobile safety application 132 includes a user interface element for selecting an amount of time to wait before triggering the alarm notification (e.g., a distress alert).

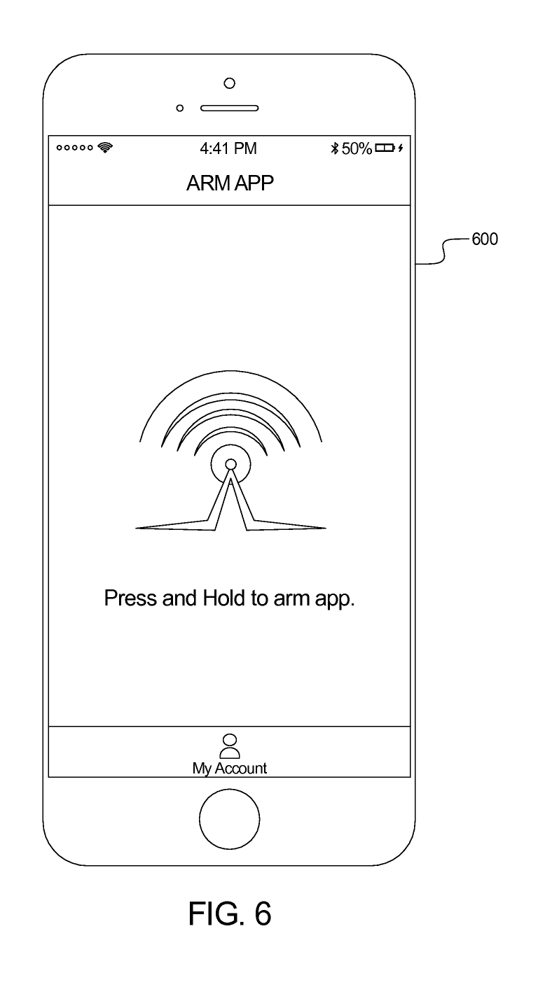

FIG. 8 illustrates an example of a drone 102 according to an example embodiment. As shown, the drone 102 includes a camera system 103, a microphone system 105, an output system 107, and an input system 109.

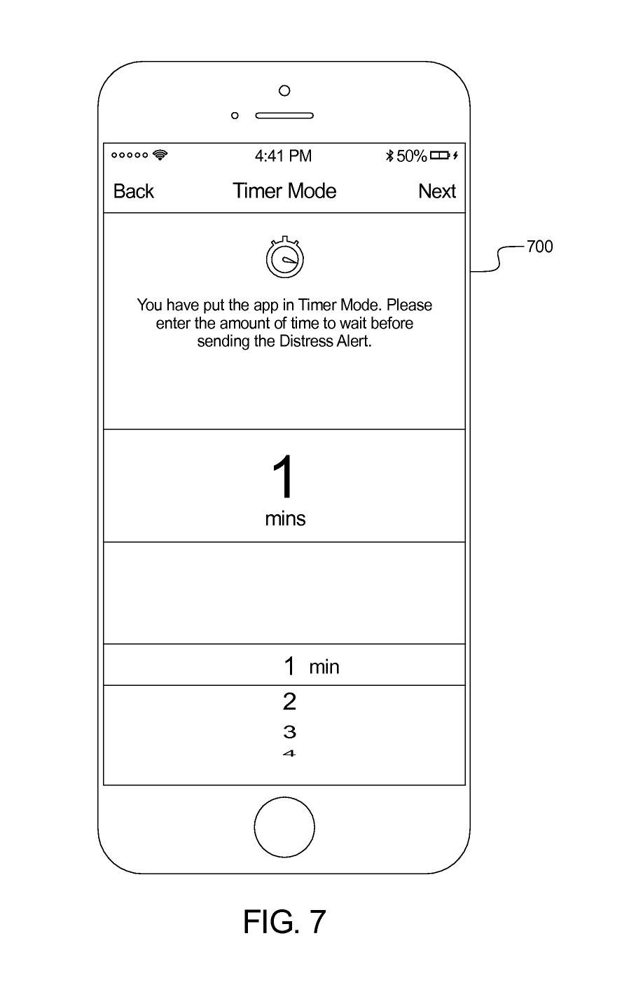

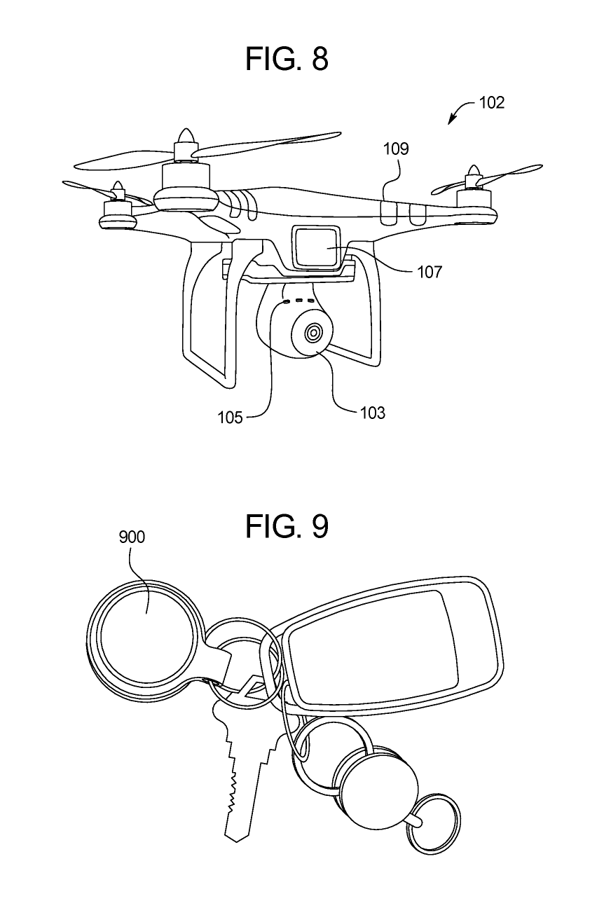

FIG. 9 illustrates a keychain including an example wearable device 900 according to an example embodiment. This example wearable device 900 is a VALRT™ wearable device. FIG. 10 illustrates another view of the example wearable device 1000 on a wristband according to an example embodiment.

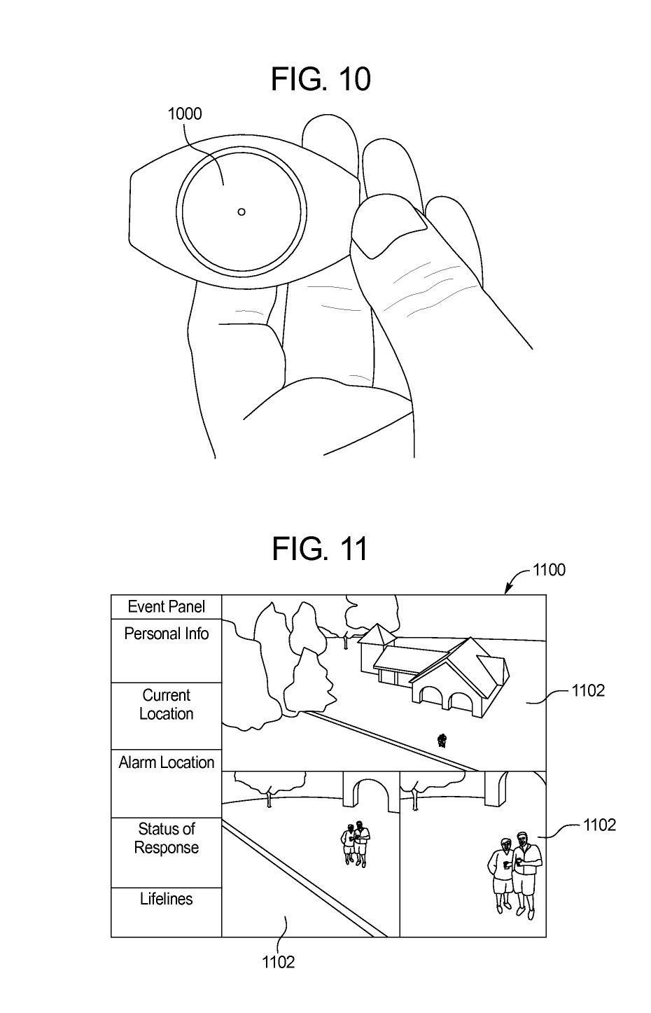

FIG. 11 illustrates a command center graphical user interface (GUI) 1100 based on an alert notification that includes one or more aerial video streams 1102 according to an example embodiment. As an example, the alarm response server 108 may display the command center GUI using the alarm response application 138 and/or the call center server 112 may display the command center GUI using the emergency dispatch application 144.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon executable instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may include, but is not limited to, magnetic storage medium (e.g., floppy diskette), optical storage medium (e.g., CD-ROM); magneto-optical storage medium, read only memory (ROM); random access memory (RAM); erasable programmable memory (e.g., EPROM and EEPROM); flash memory; or other types of medium suitable for storing electronic executable instructions.

The description above includes example systems, methods, techniques, instruction sequences, and/or computer program products that embody techniques of the present disclosure. However, it is understood that the described disclosure may be practiced without these specific details.

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A system comprising:
at least one processor to:
receive identifying information and transmit the identifying information to an alarm response server from a mobile computing device;
receive, by the mobile computing device, a unique identifier that identifies the identifying information in a database associated with the alarm response server;
receive a trigger of an alarm notification by one of a wearable device and the mobile computing device;
determine a current location of the mobile computing device;
transmit an alarm notification message to the alarm response server, the alarm notification message including the current location of the mobile computing device and the unique identifier; and
transmit the current location of the mobile computing device to at least one drone.

2. The system of claim 1, the at least one processor further to:
receive monitoring information from the at least one drone, the monitoring information comprising at least one of video, audio, and photographic information; and
store the monitoring information in the database and associate the monitoring information with the unique identifier.

3. The system of claim 1, wherein the alarm response server determines at least one public safety answering point (PSAP) based on at least one of the current location of the mobile computing device and a safety score.

4. The system of claim 1, wherein the alarm response server notifies the mobile computing device to determine whether the alarm notification is a false alarm.

5. The system of claim 1, wherein the alarm response server sends the identifying information and the current location of the mobile computing device to a call center server and to at least one lifeline.

6. The system of claim 1, wherein the alarm notification is triggered by one of a hardware button of the wearable device, a touch screen of the wearable device, a microphone of the wearable device, an accelerometer of the wearable device, a gyroscope of the wearable device, a fingerprint recognition device of the wearable device, a retina scanner of the wearable device, and a heart rate monitor of the wearable device.

7. The system of claim 1, the at least one processor further to pair the wearable device with the mobile computing device.

8. A system comprising:
a mobile device including a processor and a communication interface;
an alarm response server including a processor and a communication interface; and
a drone including a processor, a communication interface, and a camera system;
wherein,
in response to an alarm condition, the alarm response server receives an alarm notification message from the mobile device including location information;
in response to receipt of the location information, the alarm response server communicates the location information to the drone; and
in response to receipt of the location information, the drone follows a route to the location, and records monitoring information at the location.

9. The system of claim 8 wherein the alarm response server sends the mobile device a unique identifier in response to receiving setup information from the mobile device.

10. The system of claim 9 wherein, in response to receiving the monitoring information from the drone, the alarm response server stores the monitoring information in an associated database and associates the monitoring information with the unique identifier.

11. The system of claim 9 wherein, in response to receiving the unique identifier and location information, the alarm response server communicates the unique identifier and location information to a call center server.

12. The system of claim 11 wherein, in response to receiving the unique identifier and location information, the call center server communicates the location information to one or more lifelines.
